(19) United States
(12) Patent Application Publication
(10) Pub. No.: US 2025/0308671 A1
(43) Pub. Date: Oct. 2, 2025

(54) EXPOSURE THERAPY IMPLEMENTATION SYSTEM USING VIRTUAL REALITY, EXPOSURE THERAPY IMPLEMENTATION METHOD USING VIRTUAL REALITY, AND EXPOSURE THERAPY IMPLEMENTATION APPLICATION SOFTWARE USING VIRTUAL REALITY

(71) Applicant: Shinichi ITO, Mitaka-shi (JP)

(72) Inventor: Shinichi ITO, Mitaka-shi (JP)

(21) Appl. No.: 19/087,600

(22) Filed: Mar. 24, 2025

(30) Foreign Application Priority Data
Mar. 26, 2024 (JP) 2024-050178
Feb. 17, 2025 (JP) 2025-023836

(51) Int. Cl. G16H 20/70; G16H 40/67; G16H 80/00

(52) U.S. Cl. CPC G16H 20/70; G16H 40/67; G16H 80/00

(57) ABSTRACT

In the present system, a content server, a therapist terminal, and a patient terminal are connected via a communication network, and the content server is functionally designed to provide suitable content to the patient by referring to counseling information or the like transmitted from the therapist terminal regarding the patient, in which an environment for the content to be provided to the patient can be set by the content server and/or the therapist terminal.

### CROSS-REFERENCE TO RELATED APPLICATIONS

The present application claims priority under 35 U. S. C. § 119 to Japanese Patent Application No. 2024-50178, filed Mar. 26, 2024, and Japanese Patent Application No. 2025-023836, filed Feb. 17, 2025. The contents of these applications are incorporated herein by reference in their entirety.

### BACKGROUND

### Technical Field

The present invention relates to an exposure therapy implementation system using virtual reality (hereinafter referred to as “VR”), an exposure therapy implementation method using virtual reality, and exposure therapy implementation application software using virtual reality.

### Background Art

Conventionally, treatment by medication and psychotherapy has been used for agoraphobia, which is a type of anxiety disorder, for example. The effectiveness of exposure therapy, which is one of psychotherapies, is widely recognized.

Exposure therapy includes VR exposure therapy using a head-mounted display (hereinafter referred to as “HMD”), for example. Many results showing that the VR exposure therapy is as effective as exposure therapy in real space have been reported.

JP 2021-192154 A discloses a therapeutic content generation system and a method therefor. This disclosure was made to enable effective psychiatric-disorder exposure therapy even in a case where the skill of the therapist is insufficient.

In this system, the therapist's terminal and the patient's terminal are connected via the Internet, and the therapist conducts exposure therapy via the patient's terminal, using the therapist's terminal in a treatment room. Specifically, the system includes: a therapist's content generation unit including: a first therapeutic content generation/reproduction unit that generates therapeutic content from a therapeutic scene and a therapeutic scenario selected by a therapist, and causes a patient to view the therapeutic content; and a first load intensity setting storage unit that stores a load intensity of the therapeutic scenario adjusted and set by the therapist depending on the state of the patient as the current load intensity of the patient into patient information; and a patient's content generation unit including: a therapeutic content selection unit that determines therapeutic content to be recommended depending on the current load intensity of the patient and a target value of the load intensity of the patient, and causes the patient to select the therapeutic content; and a second load intensity setting storage unit that acquires a state of the patient, and sets and stores the current load intensity of the patient information about the patient in accordance with the acquired state.

JP 2022-538996 W discloses a virtual reality therapy system for providing psychotherapy to a patient. This is designed to present, to the patient, a set of therapeutic scenarios including an interaction task in a virtual environment.

Specifically, a patient character interacts with a task character in a virtual environment, the characteristics of the interaction between the patient character and the task character are measured to determine a psychological anxiety state parameter, task performance is monitored, and VR feedback including one or both of a verbal prompt and a visual prompt in the virtual environment using a coach character is provided in response to the psychological anxiety state parameter and/or the performance.

JP 2022-65576 A discloses a program, a method, an information processing device, and a system for appropriately recording information necessary for treatment of phobia. This is designed to provide a technology more suitable for treatment of phobia, by associating a feeling of fear that does not appear in vital data but is felt by the patient with time-series information in the form of a video image and sound including a stimulus of fear, and then recording the feeling of fear.

In other words, functions are configured so that a video image and sound including a stimulus of fear about which a first user as the patient feels fear is presented to the first user, and information actively expressed by the first user viewing the video image and sound through an operation by the first user or a second user as the therapist is associated with time-series information about the video image and sound and is then recorded.

There are a considerable number of persons who wish to try exposure therapy (such a person will be hereinafter referred to as a “patient”) to alleviate and cure anxiety disorder or the like including agoraphobia. However, receiving exposure therapy in real space imposes high financial, physical, and psychological hurdles, and is not easy for a patient. The fact is that it is very difficult for a patient to go to a place for therapy to begin with.

Furthermore, it is not easy for a therapist to determine and consider a symptom, a disease state, a mental state, or the like (hereinafter referred to as “a symptom or the like”) of a patient, and immediately prepare an appropriate environment to conduct exposure therapy.

### SUMMARY

According to one aspect of the present invention, a configuration includes: a counseling information generation unit having a function of generating counseling information regarding a patient, and transmitting the counseling information to the outside via a communication network; a VR content information adjustment unit that is connected to the communication network, and has a function of adjusting held content information formed with virtual reality by referring to the counseling information transmitted from the counseling information generation unit, and transmitting the adjusted content information to the outside via the communication network; a VR content information provision unit having a function of receiving the adjusted content information transmitted from the VR content information adjustment unit, and providing the content information to the patient; a reaction information detection unit having a function of detecting reaction information about the patient with respect to the content information provided by the VR content information provision unit, and transmitting the reaction information to the outside via the communication network; and a recommendation information generation unit having a function of receiving the reaction information transmitted from the reaction information detection unit via the communication network, evaluating the reaction information or further adjusting the content information by referring to the reaction information, and transmitting the content information as recommendation information to the outside via the communication network.

According to one aspect of the present invention, an exposure therapy implementation method using virtual reality is implemented by a patient terminal that provides content information formed with virtual reality to a patient, a therapist terminal that is connected to the patient terminal and is capable of controlling the content information, and a VR content management device that is connected to the therapist terminal and the patient terminal via a communication network and manages and stores the content information, the exposure therapy implementation method includes: by the therapist terminal, transmitting counseling information regarding the patient to the VR content management device via the communication network; by the VR content management device, receiving the counseling information transmitted via the communication network, adjusting the content information to be suitable for the patient by referring to the counseling information, and transmitting the adjusted content information to the patient terminal and the therapist terminal via the communication network; by the patient terminal, receiving the transmitted adjusted content information, and providing the content information to the patient; by one of the patient terminal or the therapist terminal, detecting reaction information about the patient with respect to the provided content information, and transmitting the reaction information to the VR content management device via the communication network, and by the VR content management device, receiving the transmitted reaction information, and generating recommendation information regarding the patient by referring to the reaction information.

According to one aspect of the present invention, application software for exposure therapy implementation using virtual reality installed in a patient terminal device is configured to: acquire counseling information regarding a patient and transmit the counseling information to a VR content management device; receive, with the VR content management device, content information obtained by adjusting the received counseling information to be suitable for the patient; provide the received content information to the patient; detect reaction information about the patient with respect to the provided content information and transmit the reaction information to the VR content management device; and receive recommendation information regarding the patient, the recommendation information being generated by referring to the reaction information received with the VR content management device.

### BRIEF DESCRIPTION OF DRAWINGS

FIG. 1 is a diagram showing a schematic configuration of an entire exposure therapy implementation system using VR according to a first embodiment of the present invention;

FIG. 2 is a diagram showing a schematic configuration of a VR content management device (a content server) of the exposure therapy implementation system according to the embodiment;

FIG. 3 is a diagram showing a schematic configuration of a therapist terminal of the exposure therapy implementation system according to the embodiment;

FIG. 4 is a diagram showing a schematic configuration of a patient terminal of the exposure therapy implementation system according to the embodiment;

FIG. 5 is a diagram showing an overall flow of an exposure therapy implementation process in the exposure therapy implementation system according to the embodiment;

FIG. 6 is a diagram showing a flow of an exposure therapy process using VR according to the embodiment;

FIG. 7 is a diagram schematically showing a table indicating an example of some of the items constituting counseling information according to the embodiment;

FIG. 8 is a diagram schematically showing a table indicating an example of some of the items constituting environment setting information and reaction information according to the embodiment;

FIG. 9 is a diagram schematically showing an example of VR content to be provided to and viewed by a patient according to the embodiment;

FIG. 10 is a diagram schematically showing an icon for controlling VR content to be provided to and viewed by a patient according to the embodiment;

FIG. 11 is a diagram showing an overall flow of an exposure therapy implementation process in an exposure therapy implementation system using VR according to a modification of the embodiment;

FIG. 12 is a diagram showing a schematic configuration of an entire exposure therapy implementation system using VR according to another modification of the embodiment;

FIG. 13 is a diagram showing a schematic configuration of an entire exposure therapy implementation system using VR according to a second embodiment of the present invention;

FIG. 14 is a diagram showing a schematic configuration of a VR content management device (a content server) of the exposure therapy implementation system according to the embodiment;

FIG. 15 is a diagram showing a schematic configuration of a patient terminal of the exposure therapy implementation system according to the embodiment;

FIG. 16 is a diagram showing an overall flow of an exposure therapy implementation process in the exposure therapy implementation system according to the embodiment; and

FIG. 17 is a diagram showing a flow of an exposure therapy process using VR according to the embodiment.

### DESCRIPTION OF THE EMBODIMENTS

The following is a description of embodiments of the present invention, with reference to the drawings.

### First Embodiment

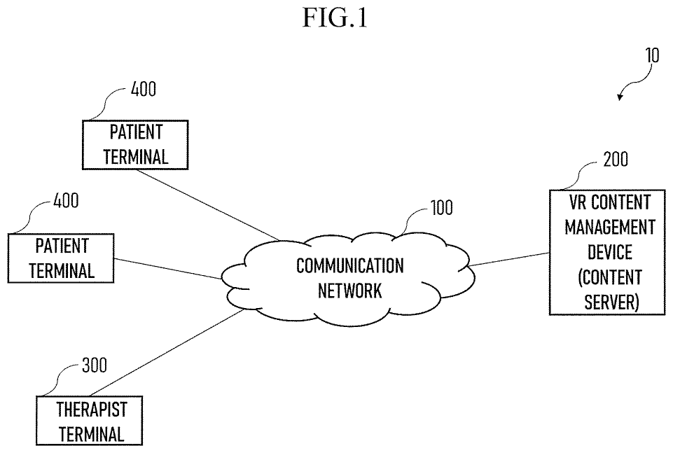

First, an exposure therapy implementation system using virtual reality (hereinafter referred to as “the present system”) 10 according to a first embodiment is formed by connecting a VR content management device (hereinafter referred to as “the content server”) 200 that manages and stores content information (hereinafter referred to as “content”) formed with virtual reality video images via a communication network 100, a therapist terminal 300, and patient terminals 400 (see FIG. 1). The content server 200 is functionally designed to provide preferred content to the patient, by referring to counseling information or the like regarding the patient sent from the therapist terminal 300. Also, the present system 10 is functionally designed so that the environment of the content to be provided to the patient can be set by the content server 200 and/or the therapist terminal 300.

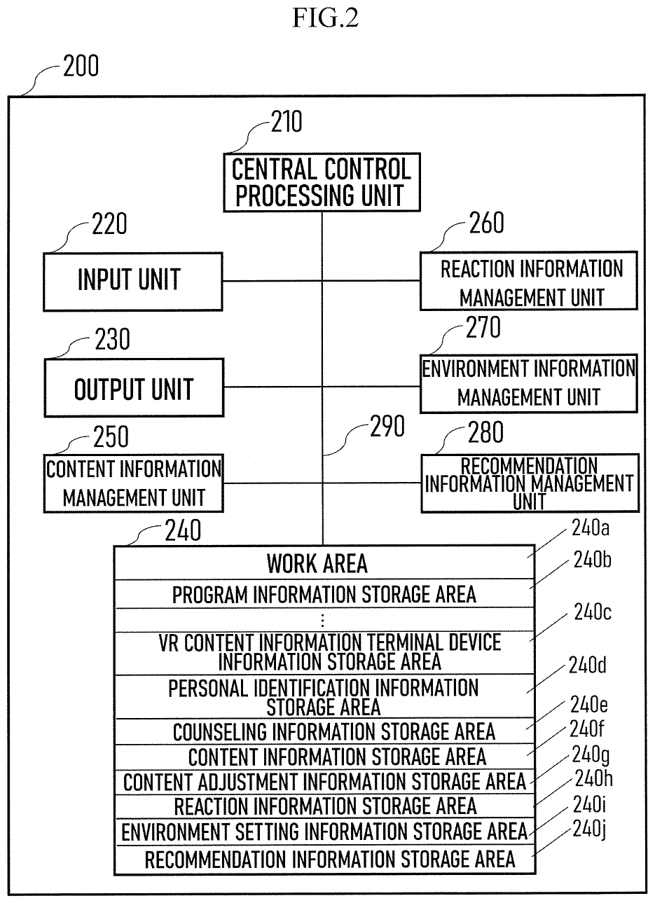

The content server 200 is a server computer having a VR content information adjustment function, and a central control processing unit 210, an input unit 220, an output unit 230, a readable/writable memory 240, a content information management unit 250, a reaction information management unit 260, an environment information management unit 270, and a recommendation information management unit 280 are connected to one another via a signal line 290 therein (see FIG. 2). Here, the signal line 290 may be wired or wireless, or may be a combination thereof.

The central control processing unit 210 has a function of controlling an overall control process in the present system 10 and a control process in the content server 200 by referring to predetermined program information stored in the memory 240. Also, the central control processing unit 210 functions to provide predetermined content to the outside via the communication network 100, by referring to a content information storage area 240f and others in the memory 240 to be described later.

Under the management of the central control processing unit 210, the input unit 220 has a function of receiving various kinds of information transmitted from the outside via the communication network 100 and storing the information into predetermined storage areas in the memory 240. For example, various kinds of information transmitted from the therapist terminal 300 are received and are stored into a predetermined storage area in the memory 240. Also, the input unit 220 has a function of storing various kinds of information and the like that are input with keys through a keyboard or the like provided therein, into a predetermined storage area in the memory 240.

Under the management of the central control processing unit 210, the output unit 230 has an output/transmission function of outputting various kinds of information and transmitting the information to the outside. For example, the output unit 230 transmits various kinds of information such as content to the therapist terminal 300 and the patient terminals 400.

The memory 240 includes a work area 240a for performing various kinds of work, and a program information storage area 240b that is a storage area storing program information to be referred to by the central control processing unit 210 for the basic control process and management.

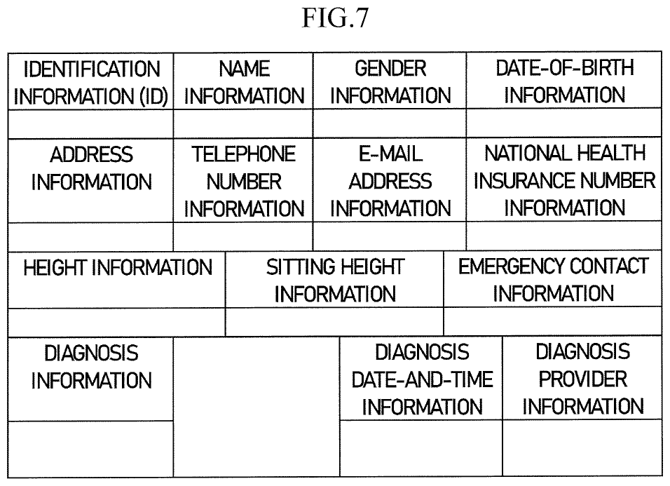

Further, the memory 240 includes the following storage areas.
A VR content information terminal device information storage area 240c that stores information related to the therapist terminal 300 and a patient terminals 400, including identification information about the terminals 300 and 400
A personal identification information storage area 240d that stores identification information and the like regarding the therapist and the patient transmitted from the therapist terminal 300, the patient terminal 400, and the like
A counseling information storage area 240e that stores counseling information (including basic medical record information about the patient (see FIG. 7), preferred setting parameter information regarding the provided content at a time of exposure therapy implementation, and subjective anxiety scale table information or anxiety hierarchy table information, hereinafter referred to as the “counseling information”) regarding the counseling performed on the patient and its result and the like transmitted from the therapist terminal 300 or the like
A content information storage area 240f that stores various kinds of content to be provided to the therapist terminal 300 and the patient terminal 400 when exposure therapy is conducted on the patient
A content adjustment information storage area 240g that stores all information related to adjustment (including non-adjustment, correction, non-correction, alteration, and non-alteration) that is to be performed or has been performed on the content at a time of exposure therapy implementation on the patient, and content adjustment program information for the adjustment
A reaction information storage area 240h that stores information regarding a reaction of the patient at a time of exposure therapy implementation (the information will be hereinafter referred to as the “reaction information”, including the patient's subjective anxiety scale table information or anxiety hierarchy table information (hereinafter referred to as “SUD information”), and biological reaction information such as an amount of perspiration, a heart rate, and a pupil response) and information (hereinafter referred to as the “evaluation information”) regarding a result of evaluation of the reaction information, and reaction evaluation information generating program information for generating the evaluation information
An environment setting information storage area 240i that stores all information (hereinafter referred to as the “environment setting information”) related to adjustment (including non-adjustment, correction, non-correction, alteration, and non-alteration, hereinafter referred to simply as the “adjustment”) that is to be performed or has been performed on the content at a time of exposure therapy implementation on the patient, and environment setting program information for performing the adjustment
A recommendation information storage area 240j that stores recommendation information (including preferred setting parameter information related to the provided content at a time of later exposure therapy implementation) that is related to the therapy and the like preferable for the patient in the future and is generated by referring to the reaction information about the patient and the environment setting information and the like, and recommendation information generating program information for generating the recommendation information

Under the management of the central control processing unit 210, the content information management unit 250 is functionally designed to refer to the VR content information terminal device information storage area 240c, the personal identification information storage area 240d, the counseling information storage area 240e, and the content information storage area 240f in the memory 240, select and adjust preferred content for the patient at the time of exposure therapy implementation, and provide the selected and adjusted content to the therapist terminal 300 and the patient terminal 400 via the output unit 230. Also, the content information management unit 250 has a function of storing the provided content associated with the identification information about the patient, into the memory 240.

That is, the content information management unit 250 performs environment setting in advance for the content to be provided depending on the degree of anxiety in agoraphobia, for example, of the patient to be subjected to exposure therapy. Specifically, when providing preferred content on the basis of the counseling information regarding the patient to be subjected to the exposure therapy, the content information management unit 250 adjusts the background, the characters, the number of characters, and the gaps (distances) between the characters and objects in the content, for example, by taking into consideration the physical features of the patient, and then provides the content to the therapist terminal 300 and the patient terminal 400.

Note that the adjustment is functionally designed to be performed on the content in accordance with a predetermined rule by referring to an adjustment table prepared in advance, on the basis the setting parameters, for example. Further, on the basis of a selection from a pull-down menu prepared in advance or a desired numerical value input, or on the basis of a recommended value from data based on generator-type AI, the reaction information about the patient, or the like, a function may be designed to suitably adjust the background, the characters, the number of characters, the distances between the characters and objects, and the like, depending on the degree of anxiety in anxiety disorder (an anxiety group and an anxiety disorder group, agoraphobia, or the like).

Under the management of the central control processing unit 210, the reaction information management unit 260 has a function of referring to the reaction information storage area 240h to refer to a table prepared in advance, and, in accordance with the predetermined rule, evaluating the reaction information about the patient' reaction to the content transmitted from the therapist terminal 300. Also, the reaction information management unit 260 has a function of storing information such as an evaluation result as the evaluation information, together with the reaction information about the patient, into the memory 240, the information being associated with the identification information about the patient in the reaction information storage area 240h.

Under the management of the central control processing unit 210, the environment information management unit 270 has a function of adjusting the setting environment of the content to be provided to the patient, by referring to the environment setting information storage area 240i. This adjustment function can be executed at any time in accordance with the predetermined rule by referring to the reaction information detected from the patient during the exposure therapy on the patient, by referring to the table prepared in advance, for example. However, the embodiment is not limited to this. Also, the environment information management unit 270 has a function of storing the information regarding the adjustment performed on the content into the memory 240, including the adjustment performed on the content by the therapist terminal 300 and the patient terminal 400, the information being associated with the identification information about the patient in the environment setting information storage area 240i.

Under the management of the central control processing unit 210, the recommendation information management unit 280 has a function of generating recommendation information regarding the therapy or the like considered suitable for the patient by referring to the recommendation information storage area 240j, the recommendation information being generated on the basis of the reaction information, the evaluation information, and the like about the patient subjected to the exposure therapy. Also, the recommendation information management unit 280 has a function of storing the generated recommendation information associated with the identification information about the patient into the recommendation information storage area 240j in the memory 240, and transmitting the recommendation information to the therapist terminal 300 via the output unit 230.

Note that, in the present embodiment, the respective functions of the content information management unit 250, the reaction information management unit 260, the environment information management unit 270, and the recommendation information management unit 280 are integrated into the single content server 200, and are managed and controlled. However, the embodiment is not limited to this, and it is needless to say that the functions may be distributed to a plurality of servers for management and control.

Further, in the present embodiment, the functions of the reaction information management unit 260 and the environment information management unit 270 are provided on the side of the content server 200, but may be provided only on the side of the therapist terminal 300 for management and control.

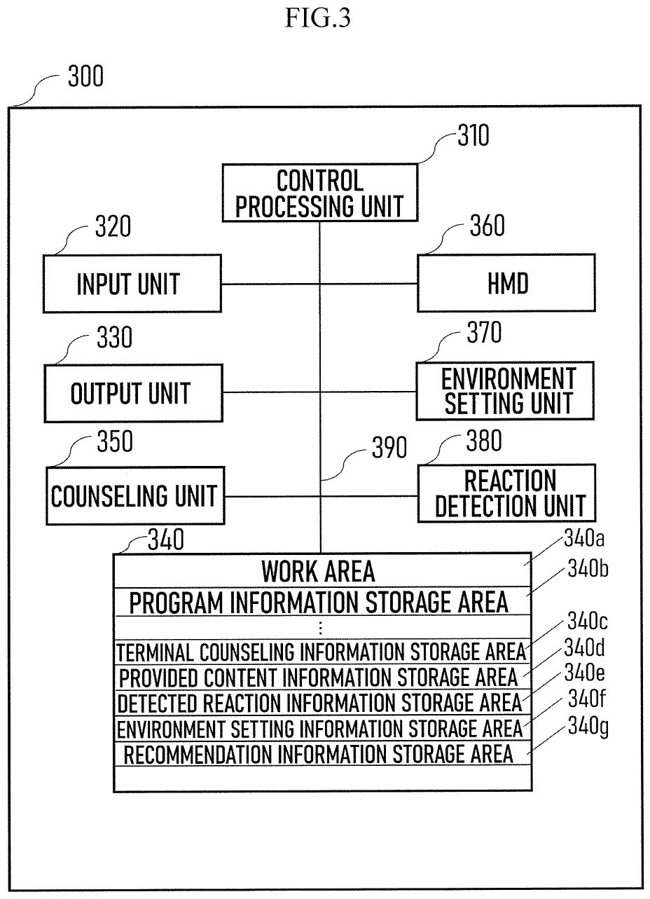

The therapist terminal 300 includes a control processing unit 310, an input unit 320, an output unit 330, a readable/writable memory 340, a counseling unit 350, an HMD 360, an environment setting unit 370, and a reaction detection unit 380, which are connected to one another via a signal line 390 (see FIG. 3). Here, the signal line 390 may be wired or wireless, or may be a combination thereof. The therapist terminal 300 is a PC, a tablet terminal, or a smartphone, for example.

Further, the therapist terminal 300 is functionally designed to be able to manage and control the patient terminal 400 at a time of exposure therapy implementation.

The control processing unit 310 has a function of controlling a control process in the therapist terminal 300 by referring to predetermined program information stored in the memory 340. Also, the control processing unit 310 has a function of stopping the exposure therapy being conducted on the side of the patient terminal 400 on the basis of emergency stop information from the patient terminal 400 or the input unit 320, or on the basis of reaction information exceeding a preset threshold. That is, the control processing unit 310 has a function of stopping viewing the content provided to the patient terminal 400.

Under the management of the control processing unit 310, the input unit 320 has a function of storing a key input from an internal keyboard, a pen input from a tablet, or input information by voice recognition, image recognition, or the like, into a predetermined storage area in the memory 340. Also, the input unit 320 has a function of storing various kinds of information transmitted from the outside via the communication network 100 into predetermined storage areas in the memory 340.

The output unit 330 has a function of displaying, printing out, or audibly outputting various kinds of information, under the management of the control processing unit 310. Also, the output unit 330 has a function of transmitting various kinds of information to the outside via the communication network 100. For example, voice information attached to provided content is audibly output.

The memory 340 includes a work area 340a for performing various kinds of work, and a program information storage area 340b that stores program information to be referred to by the control processing unit 310 to perform various kinds of control processes and management.

Further, the memory 340 includes the following storage areas.
A terminal counseling information storage area 340c for storing counseling information generated by counseling conducted on the patient on the side of the therapist terminal 300, and counseling information generation program information for generating the counseling information
A provided content information storage area 340d for storing content transmitted from the content server 200
A detected reaction information storage area 340e that stores the patient's reaction detected at a time of exposure therapy implementation as reaction information, and stores reaction detection program information for detecting a reaction
An environment setting information storage area 340f that stores environment setting information generated on the side of the content server 200, information regarding the environment setting to be performed on content on the side of the therapist terminal 300, and environment setting program information for the environment setting
A recommendation information storage area 340g that stores recommendation information generated on the side of the content server 200

Under the management of the control processing unit 310, the counseling unit 350 has a function of generating the counseling information regarding the patient by referring to the terminal counseling information storage area 340c, and storing the generated counseling information into the terminal counseling information storage area 340c. Also, the counseling unit 350 has a function of transmitting the generated counseling information to the outside via the output unit 330.

Under the management of the control processing unit 310, the HMD 360 is detachably attached to the patient, and has a function of allowing the patient to view the content provided from the content server 200 in a VR space.

Note that, in the present embodiment, the HMD 360 is integrally provided as part of a function of the therapist terminal 300. However, the embodiment is not limited to this, and, for example, the HMD 360 may be designed to be detachably connected, as appropriate, to the therapist terminal 300 via the signal line 390 only when the HMD 360 is used. Also, the therapist terminal 300 is not necessarily include the HMD 360, and only the patient terminals 400 may have the functions of an HMD.

Under the management of the control processing unit 310, the environment setting unit 370 has a function of referring to the environment setting information storage area 340f, and performing environment setting on the content to be provided to the patient from the HMD 360 or an HMD 450 to be described later. Further, the environment setting unit 370 has a function of storing information such as implementation or non-implementation of environment setting and its result or the like associated with the identification information about the patient, into the environment setting information storage area 340f.

For example, the environment setting unit 370 can adjust the environment so that the background, the characters, the number of characters, the gaps (distances) between the characters and objects in the provided content, and the height, sitting height, eye level, sound volume, and the like of the patient are optimal or suitable for the patient during the exposure therapy being conducted on the patient. Information about the result of the adjustment (including non-adjustment) is then associated with the patient, and is stored into the environment setting information storage area 340f.

Note that the adjustment is performed by inputting a selection from a pull-down menu prepared in advance or a desired numerical value, or by referring to a recommended value from data formed with generator-type AI, the reaction information about the patient, and the like (including non-adjustment, correction, non-correction, alteration, and non-alteration).

Under the management of the control processing unit 310, the reaction detection unit 380 has a function of detecting the patient's reaction to the content provided at a time of exposure therapy implementation by referring to the reaction detection program information in the detected reaction information storage area 340e, and storing information regarding the detected reaction as the reaction information into the detected reaction information storage area 340e, the information being associated with the identification information about the patient. Also, the reaction detection unit 380 has a function of transmitting the reaction information to the outside via the output unit 330. Further, the reaction detection unit 380 has a function of urgently stopping the viewing of the content provided to the patient under a predetermined condition.

Note that the reaction detection unit 380 is functionally designed to evaluate the detected reaction information, store the evaluation result information added to the reaction information into the detected reaction information storage area 340e, and transmit the evaluation result information to the outside via the output unit 330.

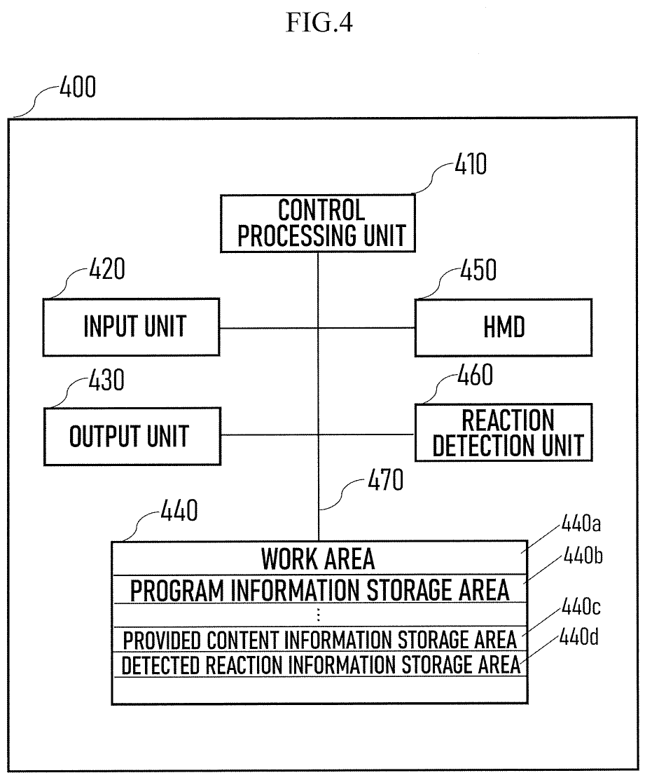

The patient terminal 400 includes a control processing unit 410, an input unit 420, an output unit 430, a readable/writable memory 440, an HMD 450, and a reaction detection unit 460, which are connected to one another via a signal line 470 (see FIG. 4). Here, the signal line 470 may be wired or wireless, or may be a combination thereof. The patient terminal 400 is a PC, a tablet terminal, or a smartphone, for example.

The patient terminal 400 is under the management and control of the therapist terminal 300 at a time of exposure therapy implementation.

The control processing unit 410 has a function of controlling a control process in the patient terminal 400 by referring to predetermined program information stored in the memory 440. Also, the control processing unit 410 has a function of stopping the exposure therapy being conducted on the side of the patient terminal 400, by referring to emergency stop information from the therapist terminal 300, or by referring to reaction information that is supplied from the reaction detection unit 460 and exceeds a preset threshold. That is, viewing/watching of the content provided to the patient is stopped.

Under the management of the control processing unit 410, the input unit 420 has a function of storing a key input from an internal keyboard, a pen input or a touch input from a tablet, or input information by voice recognition, image recognition, or the like, into a predetermined storage area in the memory 440. Also, the input unit 420 has a function of storing various kinds of information transmitted from the outside via the communication network 100 into predetermined storage areas in the memory 440.

The output unit 430 has a function of displaying, printing out, or audibly outputting various kinds of information, and a function of transmitting various kinds of information to the outside via the communication network 100, under the management of the control processing unit 410. Also, the output unit 430 can audibly output voice information attached to the provided content.

The memory 440 includes a work area 440a for performing various kinds of work, and a program information storage area 440b that stores program information to be referred to by the control processing unit 410 to perform various kinds of control processes and management.

Further, the memory 440 includes the following storage areas.
A provided content information storage area 440c for storing content transmitted from the content server 200 and content adjusted by the therapist terminal 300
A detected reaction information storage area 440d that stores the patient's reaction detected at a time of exposure therapy implementation as reaction information, and stores reaction detection program information for detecting a reaction

Under the management of the control processing unit 410, the HMD 450 has a function of outputting a video image and outputting sound, and has a function of allowing the patient to view/watch the content provided from the content server 200 in a virtual reality space, being detachably attached to the patient.

Note that, in the present embodiment, the HMD 450 is integrally provided as part of a function of the patient terminal 400. However, the embodiment is not limited to this, and for example, the HMD 450 may be functionally designed to have the functions of the patient terminal 400. Further, the functions may be designed so that the voice that is output as appropriate via the output unit 430 at a time of exposure therapy implementation is output only from the HMD 450.

Under the management of the control processing unit 410, the reaction detection unit 460 has a function of detecting the patient's reaction to the content provided at a time of exposure therapy implementation by referring to the reaction detection program information in the detected reaction information storage area 440d, and storing information regarding the detected reaction as the reaction information into the detected reaction information storage area 440d, the information being associated with the patient. Also, the reaction detection unit 460 has a function of transmitting the reaction information to the outside via the output unit 430.

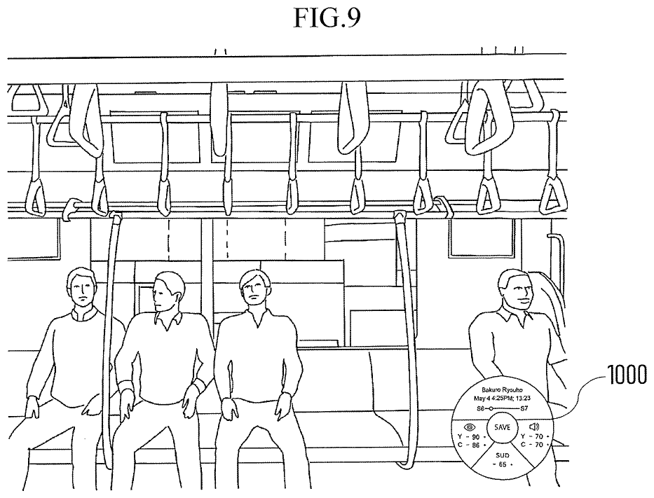

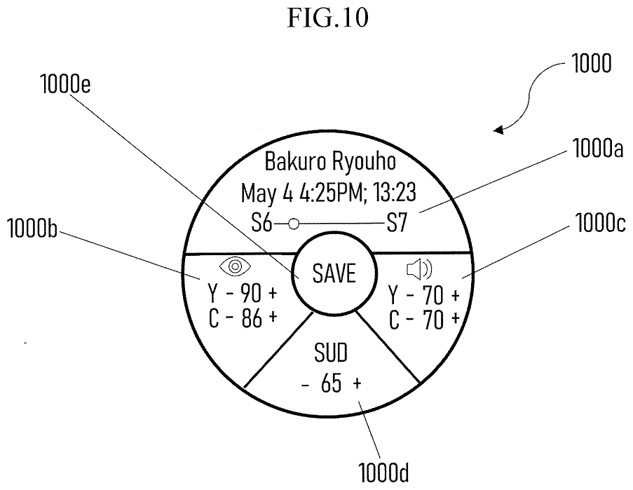

As for the above functional configuration, an example of the content to be viewed by the patient at a time of exposure therapy implementation is now described with reference to FIGS. 9 and 10.

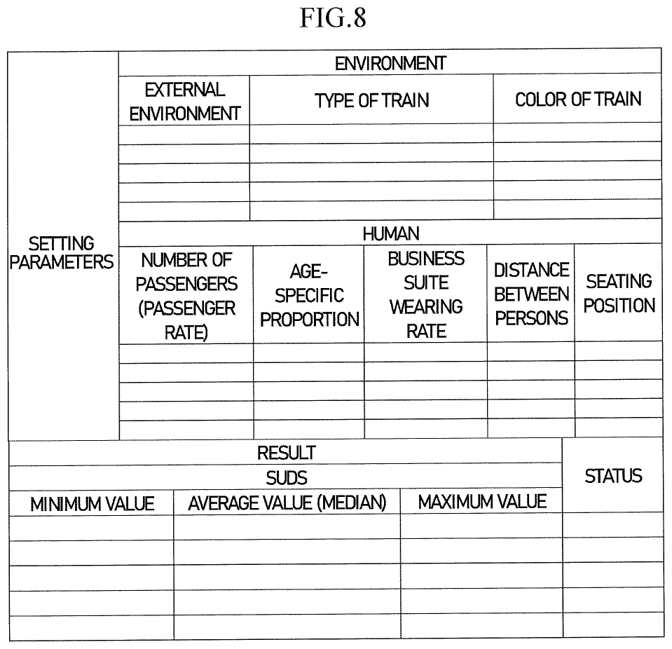

The content is based on a situation where the patient gets on a train in a VR space. For example, the following items are set and adjusted as parameters for the environments inside and outside the train the patient is to take (see a setting parameter column in FIG. 8). The setting and adjustment are performed with a selection from a pull-down menu prepared in advance, an input of a desired numerical value, a recommended value from data formed with generator-type AI, reaction information about the patient, or the like, but are not limited to this.
An external environment in which the train travels (underground, in a tunnel, in a town, in a mountain, along a beach, on an iron bridge, or the like)
The type of the train (local, rapid, express, or the like)
The color of the train (blue, yellow, orange, green, or the like)
The number of passengers/the boarding rate (the average at each station±X persons/Y %, X and Y being set as desired)
The age-specific rates among the passengers in the train compartment (the proportions of children, adults, and elderly)
The business suit wearing rate among the passenger in the train compartment (optional)
The distances between passengers/the distances between persons in the train compartment (long, short, medium, or zero)
The seating position in the train compartment (the right end, the right side, the center, the left side, the left end)

Note that the items to be set as the parameters are not limited to the above, but may be items other than the above and can be changed if necessary.

Other than the above, it is of course possible to customize content based on a situation where the patient gets on a train in a VR space and set the content as parameters as follows.
An image of each person is independently captured in advance, and the captured image is divided into layers, so that each image is subjected to synthesis processing by superimposition or the like and is adjusted.
A change to a desired number of persons can be made.
The position of the point of view of the patient is unchangeable, or is changeable.
The sound source generation position is changeable.
The parameters for the sound source and volume are adjustable.

Further, an icon 1000 having the following functions is displayed in the content to be viewed.
A function of displaying the name of the patient, date and time, the elapsed time from the start of exposure therapy implementation, and the train travel status in a progress information display area 1000a
A function of adjusting the eye levels of the patient and the therapist via an eye level adjustment area 1000b (C: for the patient, Y: for the therapist)
A function of sound volume adjustment via a sound volume adjustment area 1000c (C: for the patient, Y: for the therapist)
A function of displaying quantified SUD information in an SUD display area 1000d
A function of storing the state indicated by the above four items via a storage area 1000e

Note that the icons to be displayed are not limited to those for the above functions, but may be icons other than the above and can be changed if necessary.

An operation of the above configuration is now described.

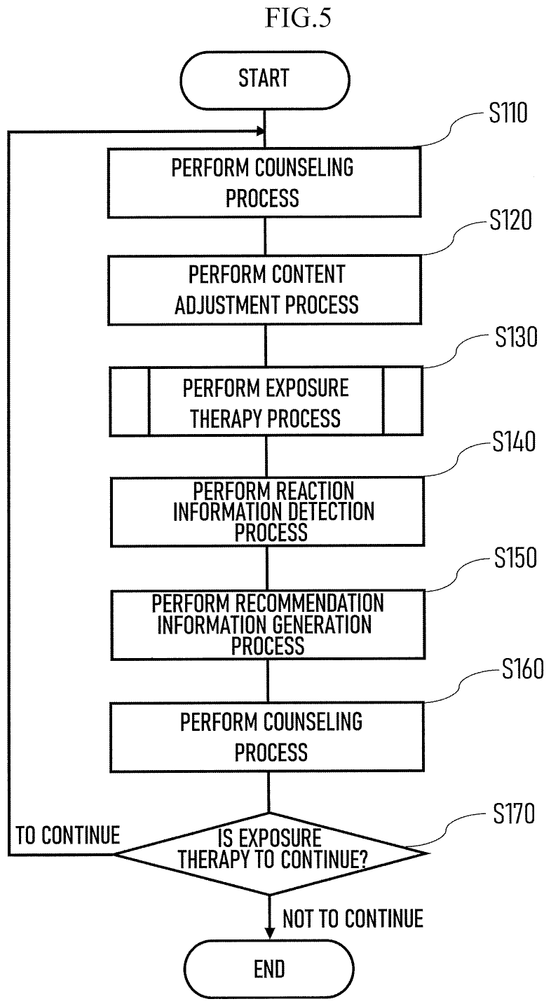

To conduct exposure therapy on a patient in the present system 10, before appropriate content is provided, predetermined counseling is first conducted on the side of the patient terminal 400 so as to generate counseling information on the side of the therapist terminal 300 (see step S110 in FIG. 5). In this case, the therapist terminal 300 acquires basic information, subjective anxiety scale table information or anxiety hierarchy table information, and the like regarding the patient from the patient terminal 400 by counseling, sets preferred parameters at the time of conducting the exposure therapy, and generates these pieces of information as the counseling information. The generated counseling information is transmitted from the therapist terminal 300 to the content server 200.

The content server 200 that has received the counseling information selects content suitable for the patient with the content information management unit 250, and adjusts the content as necessary (step S120). The selected and adjusted content is then transmitted to the therapist terminal 300 and the patient terminal 400.

The patient terminal 400 that has received the content causes the patient to view the content via the HMD 450, so that the exposure therapy is conducted under the management of the therapist terminal 300 (step S130).

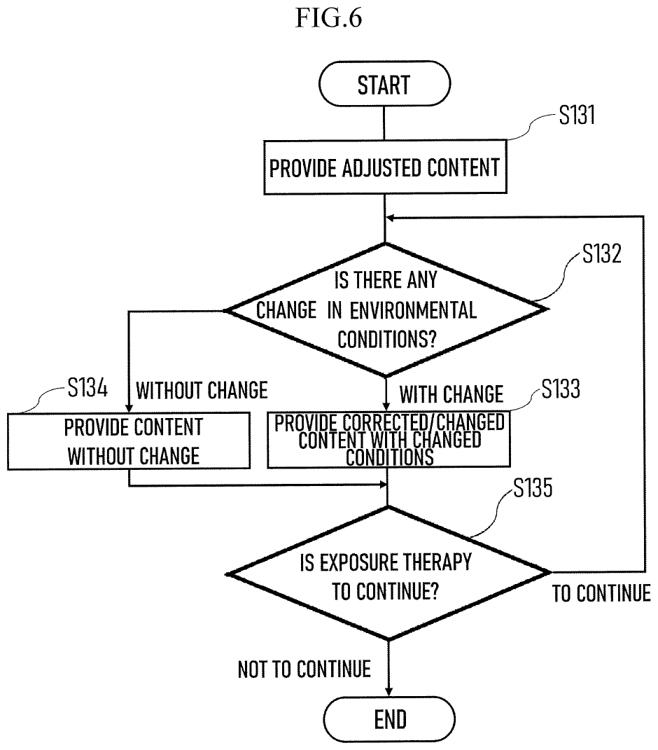

When the exposure therapy is started (see step S131 in FIG. 6), the environmental conditions of the content can be unilaterally adjusted by the environment setting unit 370 depending on the status of reaction of the patient to the content as appropriate, or without depending on the reaction status (step S132). That is, in a case where the environmental conditions are adjusted (“with change” in step S132), the patient immediately views the content under the adjusted environment (step S133). In a case where the environmental conditions are not adjusted (“without change” in step S132), the patient continues to view the content without any change (step S134).

The environmental conditions can be adjusted by the environment setting unit 370 as appropriate, while the exposure therapy is being conducted (“to continue” in step S135). Further, the eye level and the sound volume can be adjusted via the icon 1000 displayed in the content, for example.

During the exposure therapy, the reaction of the patient to the content being viewed is detected by the reaction detection units 380 and 460, and reaction information is generated. The generated reaction information is transmitted to the content server 200 (step S140).

The content server 200 refers to the reaction information and the like received by the recommendation information management unit 280, generates recommendation information suitable for the patient, and conducts a counseling. The generated recommendation information and counseling information are transmitted from the content server 200 to the therapist terminal 300, to be utilized in the next exposure therapy (steps S150 and S160).

In a case where the exposure therapy is performed next time (“to continue” in step S170), the therapist terminal 300 refers to the received recommendation information and the like, to generate new counseling information regarding the patient on the side of the patient terminal 400.

According to the above embodiment, it is possible to conduct all the exposure therapy online in real time under an environment suitable for the patient, under the management of the therapist terminal 300, and it is possible to achieve an excellent effect that the patient can receive a finely adjusted exposure therapy.

Also, according to the above embodiment, financial, physical, and psychological hurdles are lowered for the patient side, and exposure therapy can be easily received continuously, which is practically useful.

Furthermore, according to the above embodiment, the system configuration is extremely simple, and thus, the environmental load is small, and the system is socially beneficial.

It is needless to say that the present invention is not limited to the above embodiment, and various modifications can be made without departing from the scope of the present invention.

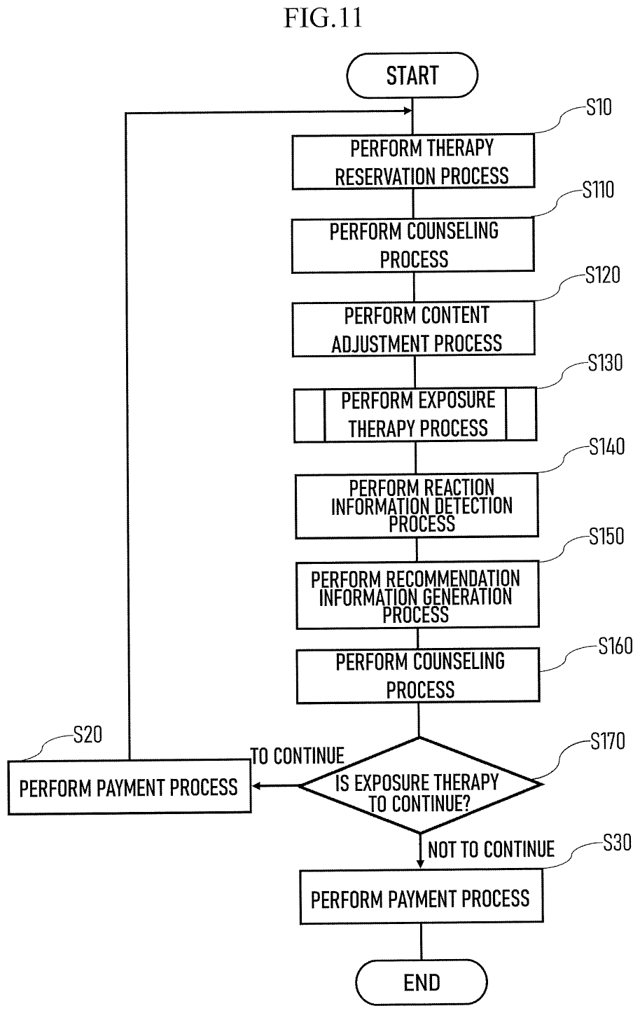

For example, FIG. 11 shows an overall flow of an exposure therapy implementation process to be performed by the present system 10 in a modification of the above embodiment. In the present modification, the same portions and/or the same functions as those of the above embodiment are denoted by the same reference numerals as those used in the above embodiment, and explanation thereof is not made herein.

In the present modification, when the patient receives exposure therapy, all of the therapy reservation and payment processes can be conducted online in real time. That is, a desired therapist, a desired date and time, and a desired payment method are input from the patient terminal 400, so that adjustment with the therapist terminal 300 is conducted at the content server 200, and a reservation process is performed (see step S10 in FIG. 11).

After the exposure therapy is conducted, a payment process is performed at the patient terminal 400 on payment instruction information transmitted from the side of the content server 200 (step S20 or step S30).

According to the present modification, in addition to the functions and effects of the above embodiment, reservation and payment processes can be performed online in real time. Thus, the load of physical movement is eliminated, and further advantageous effects can be expected both mentally and financially.

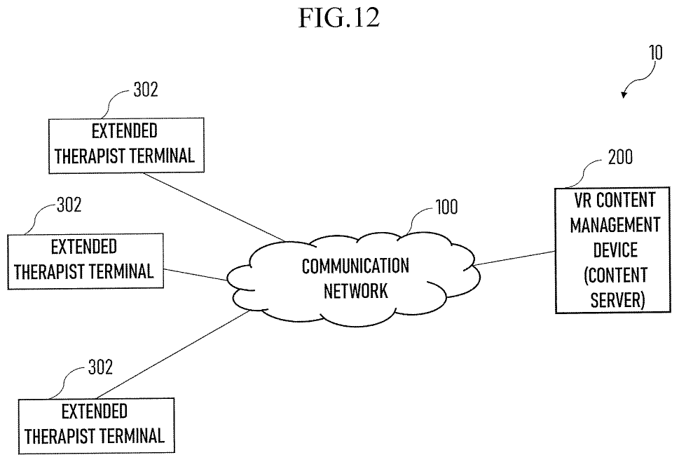

FIG. 12 is a diagram showing a schematic configuration of the entire present system according to another modification. As described above, the same portions and/or the same functions as those of the above embodiment and modification are denoted by the same reference numerals as those used in the above embodiment and modification, and explanation thereof is not made herein.

In the present modification, the functions of the therapist terminal 300 and the patient terminal 400 are integrated into one terminal (an extended therapist terminal 302). The extended therapist terminal 302 has a functional configuration that is obtained by adding the functional components included only in the patient terminal 400 shown in FIG. 4 to the functional components of the therapist terminal 300 shown in FIG. 3.

### Second Embodiment

Next, a second embodiment of the present invention is described with reference to FIGS. 7 to 10 and FIGS. 13 to 17. The same portions and the same functions as those of the first embodiment are denoted by the same reference numerals as those used in the first embodiment, and explanation of some of those portions and functions is not made herein.

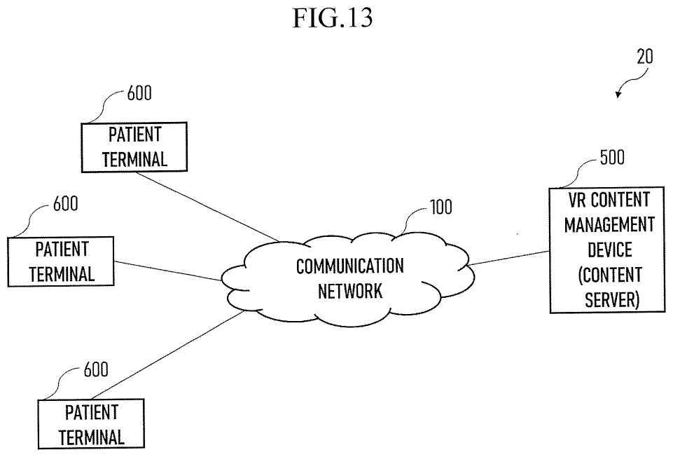

An exposure therapy implementation system using virtual reality (hereinafter referred to as “the present system”) 20 according to the present embodiment is formed by connecting a VR content management device (hereinafter referred to as “the content server”) 500 that manages and stores content information (hereinafter referred to as “content”) formed with virtual reality video images via a communication network 100, and patient terminals 600 (see FIG. 13). The content server 500 is functionally designed to provide preferred content to a patient, by referring to counseling information and the like regarding the patient transmitted from the patient terminal 600. Also, the present system 20 is functionally designed so that the environment of the content to be provided to the patient can be set by the content server 500 and the patient terminal 600.

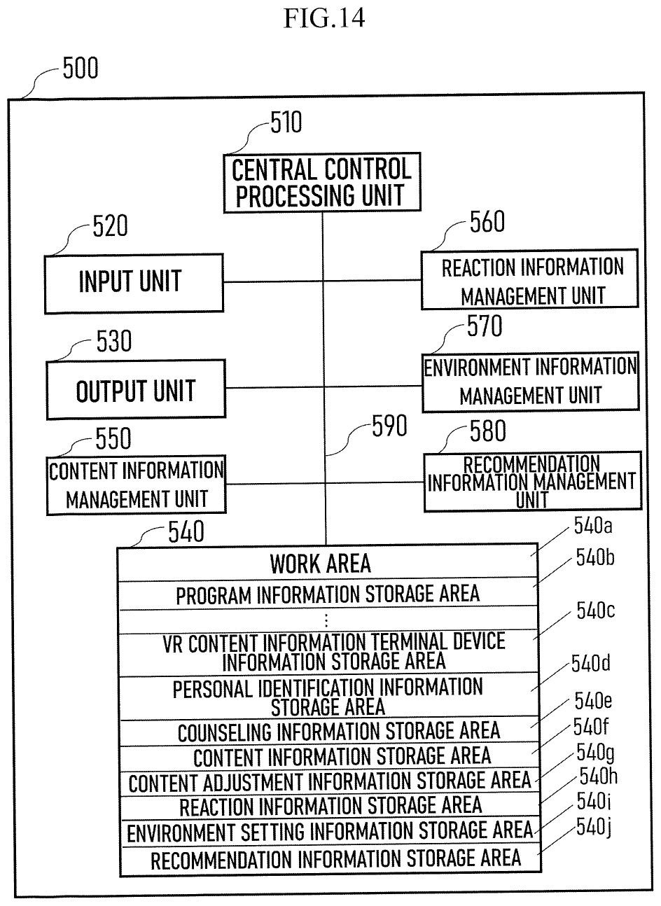

The content server 500 is a server computer having a VR content information adjustment function, and a central control processing unit 510, an input unit 520, an output unit 530, a readable/writable memory 540, a content information management unit 550, a reaction information management unit 560, an environment information management unit 570, and a recommendation information management unit 580 are connected to one another via a signal line 590 therein (see FIG. 14). Here, the signal line 590 may be wired or wireless, or may be a combination thereof.

The central control processing unit 510 has a function of controlling an overall control process in the present system 20 and a control process in the content server 500 by referring to predetermined program information stored in the memory 540. Also, the central control processing unit 510 functions to provide predetermined content to the outside via the communication network 100, by referring to a content information storage area 540f and others in the memory 540.

Under the management of the central control processing unit 510, the input unit 520 has a function of receiving various kinds of information transmitted from the outside via the communication network 100 and storing the information into predetermined storage areas in the memory 540. For example, various kinds of information transmitted from the patient terminal 600 are received and are stored into a predetermined storage area in the memory 540. Also, the input unit 520 has a function of storing various kinds of information and the like that are input with keys through a keyboard or the like provided therein, into a predetermined storage area in the memory 540.

Under the management of the central control processing unit 510, the output unit 530 has an output/transmission function of outputting various kinds of information and transmitting the information to the outside. For example, the output unit 530 transmits various kinds of information such as content to the patient terminal 600.

The memory 540 includes a work area 540a for performing various kinds of work, and a program information storage area 540b that is a storage area storing program information to be referred to by the central control processing unit 510 for the basic control process and management.

Further, the memory 540 includes the following storage areas.
A VR content information terminal device information storage area 540c that stores information related to a patient terminal 600, including identification information about the terminal 600
A personal identification information storage area 540d that stores identification information and the like regarding the patient transmitted from the patient terminal 600 and the like
A counseling information storage area 540e that stores counseling information (including basic medical record information about the patient (see FIG. 7), preferred setting parameter information regarding the provided content at a time of exposure therapy implementation, and subjective anxiety scale table information or anxiety hierarchy table information, hereinafter referred to as the “counseling information”) regarding the counseling performed on the patient and its result and the like
A content information storage area 540f that stores various kinds of content to be provided to the patient terminal 600 when exposure therapy is conducted on the patient
A content adjustment information storage area 540g that stores all information related to adjustment (including non-adjustment, correction, non-correction, alteration, and non-alteration) that is to be performed or has been performed on the content at a time of exposure therapy implementation on the patient, and content adjustment program information for the adjustment
A reaction information storage area 540h that stores information regarding a reaction of the patient at a time of exposure therapy implementation (the information will be hereinafter referred to as the “reaction information”, including the patient's subjective anxiety scale table information or anxiety hierarchy table information (hereinafter referred to as “SUD information”), and biological reaction information such as an amount of perspiration, a heart rate, and a pupil response) and information (hereinafter referred to as the “evaluation information”) regarding a result of evaluation of the reaction information, and reaction evaluation information generating program information for generating the evaluation information
An environment setting information storage area 540i that stores all information (hereinafter referred to as the “environment setting information”) related to adjustment (including non-adjustment, correction, non-correction, alteration, and non-alteration, hereinafter referred to simply as the “adjustment”) that is to be performed or has been performed on the content at a time of exposure therapy implementation on the patient, and environment setting program information for performing the adjustment
A recommendation information storage area 540j that stores recommendation information (including preferred setting parameter information related to the provided content at a time of later exposure therapy implementation) that is related to the therapy and the like preferable for the patient in the future (next time and later) and is generated by referring to the reaction information about the patient and the environment setting information and the like, and recommendation information generating program information for generating the recommendation information

Under the management of the central control processing unit 510, the content information management unit 550 is functionally designed to refer to the VR content information terminal device information storage area 540c, the personal identification information storage area 540d, the counseling information storage area 540e, and the content information storage area 540f in the memory 540, select and adjust preferred content for the patient at the time of exposure therapy implementation, and provide the selected and adjusted content to the patient terminal 600 via the output unit 530. Also, the content information management unit 550 has a function of storing the provided content associated with the identification information about the patient, into the memory 540.

That is, the content information management unit 550 performs environment setting in advance for the content to be provided depending on the degree of anxiety in agoraphobia, for example, of the patient to be subjected to exposure therapy. Specifically, when providing preferred content on the basis of the counseling information regarding the patient to be subjected to the exposure therapy, the content information management unit 550 adjusts the background, the characters, the number of characters, and the gaps (distances) between the characters and objects in the content, for example, by taking into consideration the physical features of the patient, and then provides the content to the patient terminal 600.

Note that the adjustment is functionally designed to be performed on the content in accordance with a predetermined rule by referring to an adjustment table prepared in advance, on the basis the setting parameters, for example. Further, on the basis of a selection from a pull-down menu prepared in advance or a desired numerical value input, or on the basis of a recommended value from data or particularly big data based on generator-type AI, the reaction information about the patient, and the like, a function may be designed to suitably adjust the background, the characters, the number of characters, the distances between the characters and objects, and the like, depending on the degree of anxiety in anxiety disorder (an anxiety group and an anxiety disorder group, agoraphobia, or the like).

Under the management of the central control processing unit 510, the reaction information management unit 560 has a function of referring to the reaction information storage area 540h to refer to a table prepared in advance, and, in accordance with the predetermined rule, evaluating the reaction information about the patient' reaction to the content transmitted from the patient terminal 600. Also, the reaction information management unit 560 has a function of storing information such as an evaluation result as the evaluation information, together with the reaction information about the patient, into the memory 540, the information being associated with the identification information about the patient in the reaction information storage area 540h.

Under the management of the central control processing unit 510, the environment information management unit 570 has a function of adjusting the setting environment of the content to be provided to the patient, by referring to the environment setting information storage area 540i. This adjustment function can be executed at any time in accordance with the predetermined rule by referring to the reaction information detected from the patient during the exposure therapy on the patient, by referring to the table prepared in advance, for example. However, the embodiment is not limited to this. Also, the environment information management unit 570 has a function of storing the information regarding the adjustment performed on the content into the memory 540, including the adjustment performed on the content by the patient terminal 600, the information being associated with the identification information about the patient in the environment setting information storage area 540i.

Under the management of the central control processing unit 510, the recommendation information management unit 580 has a function of generating recommendation information regarding the therapy or the like considered suitable for the patient by referring to the recommendation information storage area 540j, the recommendation information being generated on the basis of the reaction information, the evaluation information, and the like about the patient subjected to the exposure therapy. Also, the recommendation information management unit 580 has a function of storing the generated recommendation information associated with the identification information about the patient into the recommendation information storage area 540j in the memory 540, and transmitting the recommendation information to the patient terminal 600 via the output unit 530.

Note that, in the present embodiment, the respective functions of the content information management unit 550, the reaction information management unit 560, the environment information management unit 570, and the recommendation information management unit 580 are integrated into the single content server 500, and are managed and controlled. However, the embodiment is not limited to this, and it is needless to say that the functions may be distributed to a plurality of servers for management and control.

Further, in the present embodiment, the functions of the reaction information management unit 560 and the environment information management unit 570 are provided on the side of the content server 500, but may be provided only on the side of the patient terminal 600 for management and control. Alternatively, management and control may be performed on both sides.

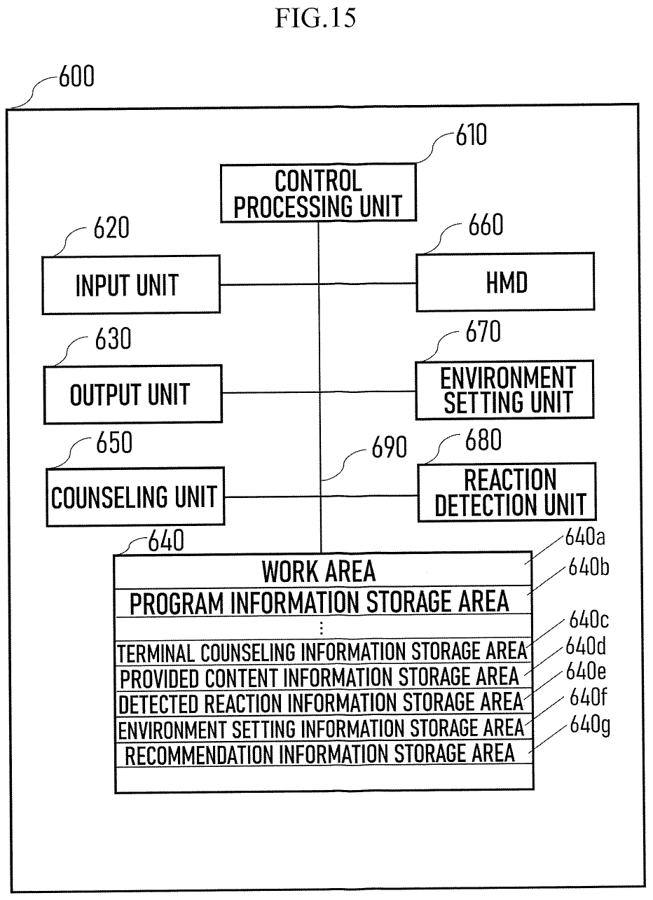

The patient terminal 600 includes a control processing unit 610, an input unit 620, an output unit 630, a readable/writable memory 640, a counseling unit 650, an HMD 660, an environment setting unit 670, and a reaction detection unit 680, which are connected to one another via a signal line 690 (see FIG. 15). Here, the signal line 690 may be wired or wireless, or may be a combination thereof. The patient terminal 600 is a PC, a tablet terminal, or a smartphone, for example.

Further, the patient terminal 600 can set a selection so as to be under the management and control of the content server 500 when exposure therapy is conducted.

The control processing unit 610 has a function of controlling a control process in the patient terminal 600 by referring to predetermined program information stored in the memory 640. Also, the control processing unit 610 has a function of stopping the exposure therapy being conducted on the side of the patient terminal 600, by referring to emergency stop information transmitted from the content server 500 or the reaction detection unit 680, or by referring to reaction information that is supplied from the reaction detection unit 680 and exceeds a preset threshold. That is, the control processing unit 610 has a function of stopping viewing/watching of the content provided to the patient.

Under the management of the control processing unit 610, the input unit 620 has a function of storing a key input from an internal keyboard, a pen input or a touch input from a tablet, or input information by voice recognition, image recognition, or the like, into a predetermined storage area in the memory 640. Also, the input unit 620 has a function of storing various kinds of information transmitted from the outside via the communication network 100 into predetermined storage areas in the memory 640.

The output unit 630 has a function of displaying, printing out, audibly outputting, or haptically outputting, by vibration (so-called haptics), various kinds of information, and a function of transmitting various kinds of information to the outside via the communication network 100, under the management of the control processing unit 610. Also, the output unit 630 can audibly output voice information attached to the provided content.

The memory 640 includes a work area 640a for performing various kinds of work, and a program information storage area 640b that stores program information to be referred to by the control processing unit 610 to perform various kinds of control processes and management.

Further, the memory 640 includes the following storage areas.
A counseling information storage area 640c for storing counseling information generated by counseling conducted on the patient on the side of the content server 500 or the patient terminal 600, and counseling information generation program information for generating the counseling information
A provided content information storage area 640d for storing content transmitted from the content server 500 (including adjusted content)
A detected reaction information storage area 640e that stores the patient's reaction detected at a time of exposure therapy implementation as reaction information, and stores reaction detection program information for detecting a reaction
An environment setting information storage area 640f that stores environment setting information generated on the side of the content server 500, information regarding the environment setting to be performed on content on the side of the patient terminal 600, and environment setting program information for the environment setting
A recommendation information storage area 640g that stores recommendation information generated on the side of the content server 500

Under the management of the control processing unit 610, the counseling unit 650 has a function of generating the counseling information regarding the patient by referring to the counseling information storage area 640c, and storing the generated counseling information into the counseling information storage area 640c. Also, the counseling unit 650 has a function of transmitting the generated counseling information to the outside via the output unit 630.

Under the management of the control processing unit 610, the HMD 660 has a function of outputting a video image and outputting sound, and has a function of allowing the patient to view/watch the content provided from the content server 500 in a virtual reality space, being detachably attached to the patient.

Note that, in the present embodiment, the HMD 660 is integrally and detachably provided as part of a function of the patient terminal 600. However, the embodiment is not limited to this, and for example, the HMD 660 may be functionally designed to have the functions of the patient terminal 600. Further, the functions may be designed so that the voice that is output as appropriate via the output unit 630 at a time of exposure therapy implementation is output only from the HMD 660.

Under the management of the control processing unit 610, the environment setting unit 670 has a function of referring to the environment setting information storage area 640f, and performing environment setting on the content to be provided to the patient from the HMD 660. Further, the environment setting unit 670 has a function of storing information such as implementation or non-implementation of environment setting and its result or the like associated with the identification information about the patient, into the environment setting information storage area 640f. Also, the environment setting unit 670 has a function of transmitting the information to the side of the content server 500.

For example, the environment setting unit 670 can adjust the environment so that the background, the characters, the number of characters, the gaps (distances) between the characters and objects in the provided content, and the height, sitting height, eye level, sound volume, and the like of the patient are optimal or suitable for the patient during the exposure therapy being conducted on the patient. Information about the result of the adjustment (including non-adjustment) is then associated with the patient, and is stored into the environment setting information storage area 640f, or the information is transmitted to the outside such as the side of the content server 500.

Note that the adjustment is performed by inputting a selection from a pull-down menu prepared in advance or a desired numerical value, or by referring to a recommended value from data or particularly big data formed with generator-type AI, the reaction information about the patient, and the like (including non-adjustment, correction, non-correction, alteration, and non-alteration).

Under the management of the control processing unit 610, the reaction detection unit 680 has a function of detecting the patient's reaction to the content provided at a time of exposure therapy implementation by referring to the reaction detection program information in the detected reaction information storage area 640e, and storing information regarding the detected reaction as the reaction information into the detected reaction information storage area 640e, the information being associated with the patient. Also, the reaction detection unit 680 has a function of transmitting the reaction information to the outside, such as the content server 500, via the output unit 630.

As for the above functional configuration, an example of the content to be viewed by the patient at a time of exposure therapy implementation is now described with reference to FIGS. 9 and 10, which have also been used in the first embodiment.

The content is based on a situation where the patient gets on a train in a VR space. For example, the following items are set and adjusted as parameters for the environments inside and outside the train the patient is to take (see a setting parameter column in FIG. 8). The setting and adjustment are performed with a selection from a pull-down menu prepared in advance, an input of a desired numerical value, a recommended value from data formed with generator-type AI, reaction information about the patient, or the like, but are not limited to this.
An external environment in which the train travels (underground, in a tunnel, in a town, in a mountain, along a beach, on an iron bridge, or the like)
The type of the train (local, rapid, express, or the like)
The color of the train (blue, yellow, orange, green, or the like)
The number of passengers/the boarding rate (the average at each station±X persons/Y %, X and Y being set as desired)
The age-specific rates among the passengers in the train compartment (the proportions of children, adults, and elderly)
The business suit wearing rate among the passenger in the train compartment (optional)
The distances between passengers/the distances between persons in the train compartment (long, short, medium, or zero)
The seating position in the train compartment (the right end, the right side, the center, the left side, the left end)

Note that the items to be set as the parameters are not limited to the above, but may be items other than the above and can be changed if necessary.

Other than the above, it is of course possible to customize content based on a situation where the patient gets on a train in a VR space and set the content as parameters as follows.
An image of each person is independently captured in advance, and the captured image is divided into layers, so that each image is subjected to synthesis processing by superimposition or the like and is adjusted.
A change to a desired number of persons can be made.
The position of the point of view of the patient is unchangeable, or is changeable.
The sound source generation position is changeable.
The parameters for the sound source and volume are adjustable.

Further, an icon 1000 having the following functions is displayed in the content to be viewed.
A function of displaying the name of the patient, date and time, the elapsed time from the start of exposure therapy implementation, and the train travel status in a progress information display area 1000a
A function of adjusting the eye levels of the patient and the therapist via an eye level adjustment area 1000b (C: for the patient, Y: for the therapist)
A function of sound volume adjustment via a sound volume adjustment area 1000c (C: for the patient, Y: for the therapist)
A function of displaying quantified SUD information in an SUD display area 1000d
A function of storing the state indicated by the above four items via a storage area 1000e

Note that the icons to be displayed are not limited to those for the above functions, but may be icons other than the above and can be changed if necessary.

An operation of the above configuration is now described.

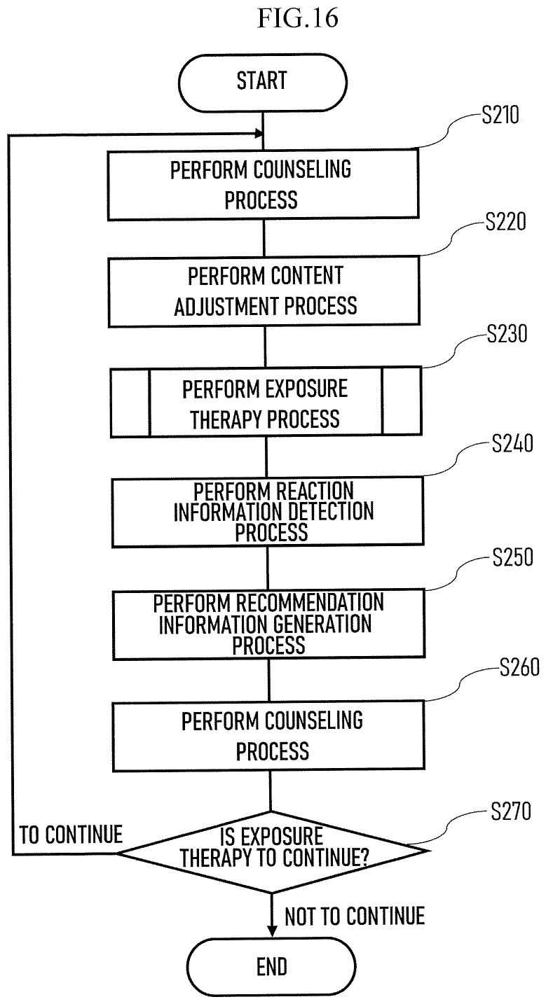

To conduct exposure therapy on a patient in the present system 20, before appropriate content is provided, predetermined counseling is first conducted on the patient so as to generate counseling information on the side of the patient terminal 600 (see step S210 in FIG. 16). In this case, basic information, subjective anxiety scale table information or anxiety hierarchy table information, and the like regarding the patient are acquired from the patient terminal 600 by counseling, preferred parameters for the time of conducting the exposure therapy is set, and these pieces of information are generated as the counseling information. The generated counseling information is transmitted from the patient terminal 600 to the content server 500.

The content server 500 that has received the counseling information selects content suitable for the patient with the content information management unit 550, and adjusts the content as necessary (step S220). The selected and adjusted content is then transmitted to the patient terminal 600.

The patient terminal 600 that has received the content causes the patient to view the content via the HMD 660, so that the exposure therapy is conducted under the management of the content server 500 with the desired settings (step S230).

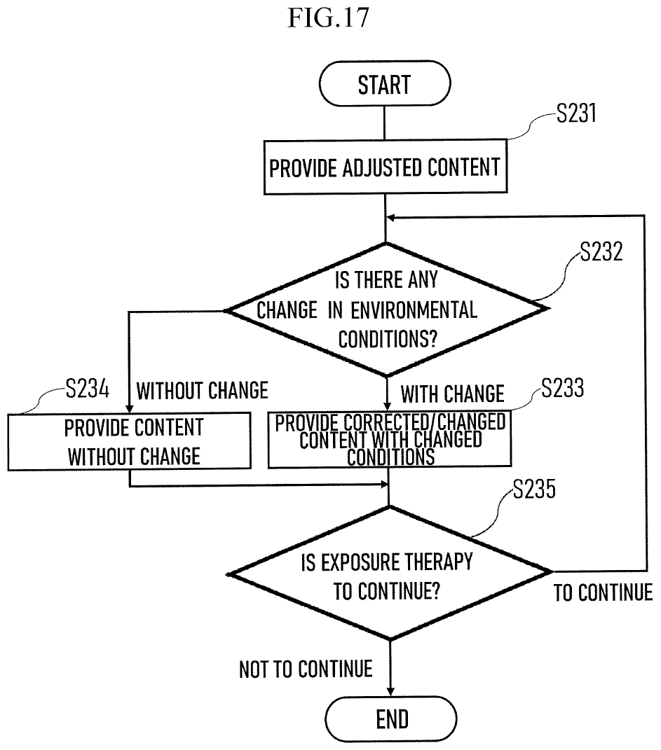

When the exposure therapy is started (see step S231 in FIG. 17), the environmental conditions of the content can be unilaterally adjusted by the environment setting unit 670 depending on the status of reaction of the patient to the content as appropriate, or without depending on the reaction status (step S232). That is, in a case where the environmental conditions are adjusted (“with change” in step S232), the patient immediately views the content under the adjusted environment (step S233). In a case where the environmental conditions are not adjusted (“without change” in step S232), the patient continues to view the content without any change (step S234).

The environmental conditions can be adjusted by the environment setting unit 670 as appropriate, while the exposure therapy is being conducted (“to continue” in step S235). Further, the eye level and the sound volume can be adjusted via the icon 1000 displayed in the content, for example.

During the exposure therapy, the reaction of the patient to the content being viewed is detected by the reaction detection unit 680, and reaction information is generated. The generated reaction information is transmitted to the content server 500 (step S240).

The content server 500 refers to the reaction information and the like received by the recommendation information management unit 580, generates recommendation information suitable for the patient, and conducts a counseling. The generated recommendation information and counseling information are transmitted from the content server 500 to the patient terminal 600, to be utilized in the next exposure therapy (steps S250 and S260).

In a case where the exposure therapy is performed next time (“to continue” in step S270), the patient terminal 600 refers to the received recommendation information and the like, to generate new counseling information regarding the patient on the side of the patient terminal 600.

According to the above embodiment, it is possible to conduct all the exposure therapy online in real time under an environment suitable for the patient, and it is possible to achieve an excellent effect that the patient can receive a finely adjusted exposure therapy.

Also, according to the above embodiment, a patient only needs to prepare a patient terminal 600. Accordingly, financial, physical, and psychological hurdles are lowered for the patient side, and exposure therapy can be easily received continuously, regardless of time and place, which is practically useful.

Furthermore, according to the above embodiment, the system configuration is extremely simple, and thus, the environmental load is small, and the system is socially beneficial.

According to the embodiment of the present invention, it is possible to provide an exposure therapy implementation system using VR, an exposure therapy implementation method using VR, and application software for exposure therapy implementation using VR that can be used online, and can provide online fine-tuned support suitable for a symptom or the like of a patient in real time.

Furthermore, according to the embodiments of the present invention, it is possible to provide an exposure therapy implementation system using VR, an exposure therapy implementation method using VR, and application software for exposure therapy implementation using VR that enable a person who wishes to receive exposure therapy to adjust financial, physical, and psychological burdens, and easily and continuously receive exposure therapy.

Furthermore, according to the embodiments of the present invention, it is possible to provide an exposure therapy implementation system using VR, an exposure therapy implementation method using VR, and application software for exposure therapy implementation using VR that require a simple configuration and impose only small environmental burdens.

Unless otherwise expressly provided, the content server, the therapist terminal, and the patient terminal are intended to include both its singular and plural forms, and its usage in any instance shall be interpreted as referring to one or more instances as contextually appropriate.

(1) A configuration includes: a counseling information generation unit having a function of generating counseling information regarding a patient, and transmitting the counseling information to the outside via a communication network; a VR content information adjustment unit that is connected to the communication network, and has a function of adjusting held content information formed with virtual reality by referring to the counseling information transmitted from the counseling information generation unit, and transmitting the adjusted content information to the outside via the communication network; a VR content information provision unit having a function of receiving the adjusted content information transmitted from the VR content information adjustment unit, and providing the content information to the patient; a reaction information detection unit having a function of detecting reaction information about the patient with respect to the content information provided by the VR content information provision unit, and transmitting the reaction information to the outside via the communication network; and a recommendation information generation unit having a function of receiving the reaction information transmitted from the reaction information detection unit via the communication network, evaluating the reaction information or further adjusting the content information by referring to the reaction information, and transmitting the content information as recommendation information to the outside via the communication network.

(2) The above configuration of (1) further includes an environment setting unit having a function of setting an environment for the content information to be provided to the patient by the VR content information provision unit.

(3) In the above configuration of (1) or (2), the counseling information generation unit has a function of generating new counseling information regarding the patient by referring to the recommendation information transmitted from the recommendation information generation unit, and transmitting the new counseling information to the outside via the communication network.

(4) An exposure therapy implementation method using virtual reality is implemented by a patient terminal that provides content information formed with virtual reality to a patient, a therapist terminal that is connected to the patient terminal and is capable of controlling the content information, and a VR content management device that is connected to the therapist terminal and the patient terminal via a communication network and manages and stores the content information, the exposure therapy implementation method includes: by the therapist terminal, transmitting counseling information regarding the patient to the VR content management device via the communication network; by the VR content management device, receiving the counseling information transmitted via the communication network, adjusting the content information to be suitable for the patient by referring to the counseling information, and transmitting the adjusted content information to the patient terminal and the therapist terminal via the communication network; by the patient terminal, receiving the transmitted adjusted content information, and providing the content information to the patient; by one of the patient terminal or the therapist terminal, detecting reaction information about the patient with respect to the provided content information, and transmitting the reaction information to the VR content management device via the communication network, and by the VR content management device, receiving the transmitted reaction information, and generating recommendation information regarding the patient by referring to the reaction information.

(5) In the above functional configuration of (4), one of the therapist terminal or the VR content management device is able to set an environment for the content information to be provided to the patient.

(6) The above functional configuration of (4) or (5) further includes: by the VR content management device, transmitting the generated recommendation information to the therapist terminal via the communication network; and by the therapist terminal, generating new counseling information regarding the patient, by referring to the recommendation information received via the communication network.

(7) Application software for exposure therapy implementation using virtual reality installed in a patient terminal device is configured to: acquire counseling information regarding a patient and transmit the counseling information to a VR content management device; receive, with the VR content management device, content information obtained by adjusting the received counseling information to be suitable for the patient; provide the received content information to the patient; detect reaction information about the patient with respect to the provided content information and transmit the reaction information to the VR content management device; and receive recommendation information regarding the patient, the recommendation information being generated by referring to the reaction information received with the VR content management device.

According to the above configuration, exposure therapy using VR can be conducted online in real time at any location, and fine-tuned support can be provided online in real time.

In addition, according to the above configuration, financial, physical, and psychological hurdles are lowered for the patient side when the exposure therapy using VR is used, and the patient can easily and continuously receive the exposure therapy.

Further, according to the above configuration, the system configuration is extremely simple, and thus, the environmental load is small, and the system is socially beneficial.

Unless otherwise expressly provided, the counseling information generation unit, the VR content information adjustment unit, the VR content information provision unit, the reaction information detection unit, the recommendation information generation unit and the environment setting unit are intended to include both its singular and plural forms, and its usage in any instance shall be interpreted as referring to one or more instances as contextually appropriate.

It is needless to say that the present invention is not limited to the above respective embodiments, and various modifications can be made to them without departing from the scope of the present invention.

What is claimed is:

1. An exposure therapy implementation system using virtual reality, comprising:
a counseling information generation unit having a function of generating counseling information regarding a patient, and transmitting the counseling information to an outside via a communication network;
a VR content information adjustment unit connected to the communication network, the VR content information adjustment unit having a function of adjusting held content information formed with virtual reality by referring to the counseling information transmitted from the counseling information generation unit, and transmitting the adjusted content information to the outside via the communication network;
a VR content information provision unit having a function of receiving the adjusted content information transmitted from the VR content information adjustment unit, and providing the content information to the patient;
a reaction information detection unit having a function of detecting reaction information about the patient with respect to the content information provided by the VR content information provision unit, and transmitting the reaction information to the outside via the communication network; and
a recommendation information generation unit having a function of receiving the reaction information transmitted from the reaction information detection unit via the communication network, evaluating the reaction information or further adjusting the content information by referring to the reaction information, and transmitting the content information as recommendation information to the outside via the communication network.

2. The exposure therapy implementation system using virtual reality according to claim 1, further comprising an environment setting unit having a function of setting an environment for the content information to be provided to the patient by the VR content information provision unit.

3. The exposure therapy implementation system using virtual reality according to claim 1, wherein the counseling information generation unit has a function of generating new counseling information regarding the patient by referring to the recommendation information transmitted from the recommendation information generation unit, and transmitting the new counseling information to the outside via the communication network.

4. The exposure therapy implementation system using virtual reality according to claim 2, wherein the counseling information generation unit has a function of generating new counseling information regarding the patient by referring to the recommendation information transmitted from the recommendation information generation unit, and transmitting the new counseling information to the outside via the communication network.

5. An exposure therapy implementation method using virtual reality formed with a patient terminal that provides content information formed with virtual reality to a patient, a therapist terminal that is connected to the patient terminal and is capable of controlling the content information, and a VR content management device that is connected to the therapist terminal and the patient terminal via a communication network and manages and stores the content information,
the exposure therapy implementation method comprising:
by the therapist terminal, transmitting counseling information regarding the patient to the VR content management device via the communication network;
by the VR content management device, receiving the counseling information transmitted via the communication network, adjusting the content information to be suitable for the patient by referring to the counseling information, and transmitting the adjusted content information to the patient terminal and the therapist terminal via the communication network;
by the patient terminal, receiving the transmitted adjusted content information, and providing the content information to the patient;
by one of the patient terminal or the therapist terminal, detecting reaction information about the patient with respect to the provided content information, and transmitting the reaction information to the VR content management device via the communication network, and
by the VR content management device, receiving the transmitted reaction information, and generating recommendation information regarding the patient by referring to the reaction information.

6. The exposure therapy implementation method using virtual reality according to claim 5, wherein one of the therapist terminal or the VR content management device is able to set an environment for the content information to be provided to the patient.

7. The exposure therapy implementation method using virtual reality according to claim 5, further comprising:
by the VR content management device, transmitting the generated recommendation information to the therapist terminal via the communication network; and
by the therapist terminal, generating new counseling information regarding the patient, by referring to the recommendation information received via the communication network.

8. The exposure therapy implementation method using virtual reality according to claim 6, further comprising:
by the VR content management device, transmitting the generated recommendation information to the therapist terminal via the communication network; and
by the therapist terminal, generating new counseling information regarding the patient, by referring to the recommendation information received via the communication network.

9. Application software for exposure therapy implementation using virtual reality installed in a patient terminal device, the application software configured to:
acquire counseling information regarding a patient and transmit the counseling information to a VR content management device;
receive, with the VR content management device, content information obtained by adjusting the received counseling information to be suitable for the patient;
provide the received content information to the patient;
detect reaction information about the patient with respect to the provided content information and transmit the reaction information to the VR content management device; and
receive recommendation information regarding the patient, the recommendation information being generated by referring to the reaction information received with the VR content management device.
